(19) United States
(12) Patent Application Publication
(10) Pub. No.: US 2026/0232909 A1
(43) Pub. Date: Aug. 13, 2026

(54) CARPULE HAVING COLLAPSIBLE CHAMBER AND USE THEREOF

(71) Applicant: PHARMAPHDII, LLC, Plantation, FL (US)

(72) Inventor: Max Arocha, Plantation, FL (US)

(73) Assignee: PHARMAPHDII, LLC, Plantation, FL (US)

(21) Appl. No.: 19/151,309

(22) PCT Filed: Jan. 26, 2024
(86) PCT No.: PCT/US2024/013237

§ 371 Date: Jul. 26, 2025

Related U.S. Application Data
(60) Provisional application No. 63/481,786, filed on Jan. 26, 2023.

(51) Int. Cl. A61M 5/28; A61J 1/06; A61M 5/24; A61M 39/00; A61M 39/24

(52) U.S. Cl. CPC A61M 5/282; A61J 1/062; A61M 5/2425; A61M 5/2448; A61M 5/2459; A61M 5/284; A61M 5/286; A61M 39/24; A61M 2039/0009; A61M 2202/0007; A61M 2202/0241; A61M 2205/0238

(57) ABSTRACT

Described is a carpule capable of being used with a conventional cartridge syringe, the carpule having a reservoir for storing drug solution, a separate collapsible chamber for storing a buffer solution, and a mixing area or a discrete mixing chamber.

### BACKGROUND OF THE INVENTION

Local anesthetics are considered some of the safest and most effective drugs in medicine for the prevention and management of pain.

Among the more commonly used local anesthetics is the amide local anesthetic, lidocaine, which is suitable for infiltration, block, and surface anesthesia. Lidocaine, or its hydrochloride salt, is commonly pre-mixed with epinephrine for use in dental medicine and is a generally acidic solution.

Injection of an acidic lidocaine solution into the tissue can result in discomfort to the patient in the form of a “stinging” sensation and pain. Reducing the acidity (raising the alkalinity or pH) of lidocaine solution can minimize the associated “sting” and pain to the patient.

Injectable lidocaine cannot, however, be premixed with a basic solution, such as sodium bicarbonate, and stored, due to the instability of the resulting neutralized solution and formation of precipitates. Accordingly, lidocaine must be buffered or alkalinized with a bicarbonate immediately prior to injection.

Mixing devices for pre-mixing anesthetic and buffer solutions remotely (away from the patient), and immediately prior to administration, are known and in common use. However, these conventional methods and devices for buffering or alkalinizing local anesthetics still have disadvantages, including:
1. Commercially available mixing devices involve a high degree of technique and require manual, on-site mixing immediately prior to filling of the syringe for administration of the buffered anesthetic;
2. On-site preparation time is greater than the actual time required to administer buffered anesthetic mixture.
3. Buffered anesthetic solution prepared in bulk results in decreased pH of the solution over time, resulting in diminished activity.
4. Freshly mixing buffered anesthetic solutions for subsequent administrations requires repeated and redundant preparation steps.

As is well known, a conventional carpule used with a cartridge syringe, or aspirating cartridge syringe, is a generally hollow tube having a rigid cylindrical side wall, usually made from glass or transparent plastic material. A conventional carpule is shaped having a shouldered proximal end which includes a fixed or capped stopper penetrable by a hypodermic transfer needle, and having at its other end—its distal end—a sliding plug or sliding stopper which is actuated and displaced by a plunger of the cartridge syringe when in use administering drug to a patient. The carpule wall and closed ends form a cavity within the carpule serving as a reservoir for containing and storing a single drug solution or buffer solution for injection using the cartridge syringe.

Although modifications and improvements have been made to conventional syringes to control the pH of the local anesthetic solution as it is administered, these technological advances have failed to address the problems and provide efficient means for mixing anesthetic and buffer immediately prior to or concomitantly with administration in a practical and easy-to-use manner in a clinical setting. For example, mixing syringes for mixing a local anesthetic and a buffering agent within the syringe barrel have been described. More recently, U.S. Pat. No. 10,420,888 describes a side-by-side dual-barrel cartridge syringe for receiving two single carpules or one dual carpule. However, these prior attempts have been unable to resolve the problems in a cost-efficient manner.

What is needed is a carpule wherein lidocaine injection can be premixed with buffer within the carpule itself, immediately prior to or concomitant with administration in a way that can overcome the known disadvantages and deficiencies and in a cost-effective manner.

### SUMMARY OF THE INVENTION

The subject invention concerns a carpule capable of being used with or in a conventional cartridge syringe or aspirating cartridge syringe. In addition to having a primary reservoir for containing and storing drug solution, the carpule of the subject invention comprises, within the reservoir, a discrete chamber for containing and storing a second solution, such as buffer solution, separate and apart from the drug solution contained within the primary reservoir. In a carpule of the subject invention, this separate chamber is collapsible.

Generally, the invention pertains to a uniquely configured carpule for use with a cartridge syringe. The carpule of the subject invention comprises a primary reservoir for containing and storing drug solution, such as an anesthetic solution, e.g., lidocaine, Septocaine™, or other conventional anesthetic solution used in medical or dental treatments, wherein the reservoir is bounded by a cylindrical or tubular carpule sidewall, a proximal end plug, and a distal sliding stopper or plunger. A discrete collapsible chamber is disposed within the reservoir, and serves to contain and store a second solution, e.g., a buffer solution, separate and apart from the solution contained within the primary reservoir. The collapsible chamber is disposed within the reservoir such that a mixing area is provided for receiving the solutions expelled from the primary reservoir and collapsible chamber, allowing for mixing of the solutions before administration to a patient.

In one embodiment, the collapsible chamber can be configured as a tube or cylinder abutting and running along an inner face of the carpule side wall. The collapsible chamber includes a sealed distal end and sealed proximal end, wherein the proximal end seal can comprise a rupturable membrane or one or more unidirectional valves which open under pressure applied when the cartridge syringe is actuated to move the sliding stopper or plunger. In this embodiment, the collapsible chamber is flattened as the sliding stopper or plunger moves proximally. The collapsible chamber thereby expels the second solution contained therein into the mixing area or mixing chamber at the proximal end of the carpule reservoir.

In another embodiment, the collapsible chamber is configured as a pleated or accordioned tubular bellows disposed along a central longitudinal axis of the carpule. This embodiment also includes a sealed distal end and sealed proximal end, wherein the proximal end seal comprises a rupturable membrane or one-way valve opened by applied pressure when the cartridge syringe is actuated. In this embodiment, the sliding stopper or plunger compresses the collapsible chamber wherein the sealed distal end of the collapsible chamber moves toward the proximal end of the collapsing chamber. The proximal end seal is ruptured, or the proximal end valve is opened, under the pressure applied to expel the second solution from the collapsible chamber, mixing with the solution within the reservoir in the mixing area or mixing chamber.

Alternatively, the mixing area comprises a discrete mixing chamber having a valved partition. In this embodiment, having a discrete mixing chamber, the valved partition comprises at least two unidirectional valves. The mixing chamber is in fluid communication with the primary reservoir through a first unidirectional valve provided in the valved partition, and the mixing chamber is in fluid communication with the collapsible chamber through a second unidirectional valve provided in the valved partition.

When a discrete mixing chamber is present, a turbulence-enhancing apparatus can be disposed therewithin. Preferably, the turbulence enhancing apparatus is not integral with the mixing chamber; rather, it is provided as a separate component.

In any embodiment described herein, any component of the carpule exposed to the drug or buffer solution can be coated with an inert, protective coating agent. Commonly used protective coatings include silicone or a fluoropolymer.

The sliding stopper or plunger provided in the carpule has a distal end side or face and a proximal side or face. The distal face is typically substantially flat or domed and the proximal face can include a recess for matingly receiving the distal end of the collapsible chamber. At the proximal end of the collapsible chamber, a conical or inverted cup-shaped member can be provided to stabilize the collapsible chamber and provide a mixing area proximal to the conical member and distal to the proximal end plug. The conical member has a distal side or face and a proximal side or face, wherein said distal face of the conical member includes a recess to matingly receive the proximal end of the collapsible chamber, and the proximal face of the conical member forms a ring which engages a shouldered internal sidewall of the carpule. Preferably, the conical member comprises at least one port or vent allowing solution expelled from the collapsible chamber to backflow and mix with solution in the reservoir.

Also included as part of the invention is a method for mixing anesthetic solution and buffer solution and concomitantly administering mixed anesthetic solution and buffer solution to a patient in need thereof. The steps of the method comprise:
providing a carpule of as described herein;
prefilling the collapsible chamber with buffer solution and prefilling the reservoir with anesthetic solution outside the collapsible chamber,
inserting the carpule into a cartridge syringe for use, and
actuating the cartridge syringe to concomitantly compress the collapsible chamber, causing mixing of the buffer solution and anesthetic solution in the mixing area, and
administering the mixed buffer solution and anesthetic solution to the patient.

The method of the invention can preferably be employed using a carpule having a collapsible chamber which is configured as a pleated or accordioned tube or cylinder. The method can preferably be carried out during a dental procedure using sodium bicarbonate as the buffer solution and using an anesthetic solution comprising lidocaine. In dental procedures, the preferred anesthetic solution is lidocaine with epinephrine.

Advantageously, the mixed buffer solution and anesthetic solution using a carpule having a collapsible chamber according to the subject invention can provide a mixed solution having a pH of about 7.4.

Also considered as part of the subject invention is a system for pre-mixing anesthetic and buffer solutions and administering the pre-mixed solutions to a patient, wherein the system comprises a conventional cartridge syringe and a carpule having a collapsible chamber as described herein. The collapsible chamber is preferably a pleated or accordioned tube or cylinder. In a system of the subject invention, the carpule can be pre-filled with buffer solution in the collapsible chamber, and pre-filled with anesthetic solution in the reservoir, but wherein the anesthetic solution is not provided within (is outside of) the collapsible chamber. Preferably the pre-filled solutions comprise sodium bicarbonate as the buffer solution and lidocaine, with or without epinephrine, as the anesthetic solution.

The collapsible chamber disposed within the reservoir of the carpule forms a lumen therewithin and contains and discretely stores buffer solution within the lumen separate and apart from anesthetic solution contained and stored within the carpule reservoir. The collapsible chamber is generally elongate and can be configured as a hollow cylinder or tube, and preferably comprises an inert collapsible plastic or polymeric material. The hollow tube or cylinder can further comprise a protective coating applied on its external surface or its internal (lumen) surface to prevent damage or deterioration when used with an acidic or alkaline substance stored within the reservoir or within the hollow tube itself.

At its distal end, the collapsible chamber can be permanently sealed to prevent and preclude contents of the collapsible chamber, i.e., buffer solution, from being expelled from the distal end thereof prior to use. At its proximal end, the collapsible chamber is sealed, but in a manner which permits the seal to be broken or otherwise unsealed, typically by applying pressure to the collapsible chamber, and allowing for the contents (buffer solution) within the lumen of the collapsible chamber to be expelled and mixed with the contents (drug solution) of the carpule main reservoir, wherein the mixture of the buffer and drug solutions is administered to the patient. For example, a rupturable membrane can be employed as the seal. Alternatively, the proximal end of the collapsible chamber can comprise a one-way valve which can serve as a seal, or is separately sealed by a rupturable membrane, wherein the one-way valve and seal opens when pressure is applied by the fluid within the collapsible chamber, permitting the contents within the collapsible chamber to be expelled, mix with the contents of the reservoir, and delivered to the patient.

In an embodiment, a carpule of the invention can also include, between the fixed or capped stopper and the sliding plug or sliding stopper, a partition positioned at a proximal end of the carpule, horizontally disposed and generally perpendicular to the longitudinal axis of the carpule, forming a cavity serving as a mixing chamber for receiving solutions from the reservoir and collapsible chamber which are distal to the mixing chamber. In one embodiment, the partition can comprise one or more unidirectional valve, wherein drug solution from the reservoir and buffer solution from the collapsible chamber are permitted to enter the mixing chamber following application of pressure to the reservoir and collapsible chamber using the syringe plunger.

In another embodiment, the partition forming the mixing chamber can be punctured using at least one puncturing needle affixed to a pushrod extending through the fixed or capped stopper of the carpule and which is operable by a pushing motion in a distal direction by a user's thumb or finger. Using two penetrating needles, a first penetrating needle can be positioned to puncture through the partition to allow drug solution from the reservoir to enter the mixing chamber and a second penetrating needle can be positioned to puncture the partition and the proximal sealed end of the collapsible chamber to permit contents (buffer solution) from the collapsible chamber to enter into the mixing chamber.

In yet another embodiment, the partition can comprise at least one port or aperture therein which can be aligned such that the proximal end of the collapsible chamber is opened, allowing fluid communication between the collapsible chamber and mixing chamber. A second port or aperture in the partition can align in a way to allow fluid communication between the reservoir and the mixing chamber. The ports can be aligned by rotating the partition using a pushrod extending through the fixed or capped stopper of the carpule and which is operable by a rotational or twisting motion by a user.

Optionally, a separate mixing chamber can be provided. When present, a mixing chamber can provide for mixing of the solutions before their administration to a patient. The cavity of the mixing chamber is bounded at its proximal end by the capped stopper, is bounded on the sides by the carpule wall or wall liner, and is bounded at its distal end by the valved partition. The valved partition is positioned at a distance away from the capped end to provide a mixing chamber volume of about 0.1-0.5 ml of fluid.

In certain embodiments having a separate mixing chamber, the valved partition comprises at least two unidirectional, or one-way, valves. A first unidirectional valve allows for unidirectional fluid communication between the carpule reservoir contents and the mixing chamber under pressure created when actuating the plunger of the syringe. A second unidirectional valve allows for fluid communication between the mixing chamber and a collapsible chamber disposed within the reservoir of the carpule. Unidirectional flow is from the distal position (away from the patient) toward the proximal position (toward the patient).

In an embodiment having a mixing area provided at the proximal end of the carpule, a single unidirectional valve can be provided to deliver contents from the collapsible chamber to the mixing area. Contents from the carpule reservoir are separated from the mixing area via a rupturable membrane, which is ruptured by pressure applied to the liquid inside the reservoir when actuating the plunger or piston of the syringe. Alternatively, the collapsible chamber can employ a pressure-sensitive rupturable membrane to hold the contents within the collapsible chamber, and deliver the contents to the mixing area upon application of pressure by actuating the syringe piston or plunger.

The collapsible chamber disposed within the reservoir of the carpule forms a lumen therewithin and contains and discretely stores buffer solution within the lumen separate and apart from anesthetic solution contained and stored within the carpule reservoir. The collapsible chamber is generally elongate and can be configured as a hollow cylinder or tube, and preferably comprises an inert collapsible plastic or polymeric material.

At its proximal end, the collapsible chamber engages the valved partition allowing for the contents within the lumen of the collapsible chamber to be in unidirectional fluid communication with the mixing chamber. The distal end of the collapsible chamber is preferably closed or sealed.

In one embodiment, the collapsible chamber is disposed within the carpule reservoir and is positioned along the inner face of the carpule reservoir wall. Actuation of the syringe plunger causes the sliding plug to engage with the collapsible chamber causing the collapsible chamber to flatten against the inner face of the carpule reservoir wall, thereby collapsing the lumen and unidirectionally pushing or squeezing out the contents of the chamber from the proximal end of the collapsible chamber as the sliding plug moves distally, and along the longitudinal axis of the reservoir. The contents of both the carpule reservoir and the collapsible chamber empty into the cavity of the mixing chamber by actuation of the syringe plunger and proximal movement of the sliding plug.

In another embodiment, the collapsible chamber can be configured as a generally tubular reservoir wherein the tubular wall is folded in a pleated or “accordion” fashion, and effectively forms a bellows which unidirectionally empties when compressed. Engagement of the proximal end of this pleated tube embodiment of the collapsible chamber with the valved partition provides for the contents of the pleated collapsible chamber to empty through the unidirectional valve and into the cavity of the mixing chamber or mixing area when the syringe plunger actuates the sliding plug or stopper.

In both embodiments of the collapsible chamber described above, actuation of the sliding plug by the syringe plunger forces anesthetic solution and buffer solution into the mixing chamber before the resulting mixture of buffered anesthetic solution flows into a hypodermic transfer needle and is administered to a patient.

It is therefore one object of the present invention to provide a system comprising a carpule for use with an aspirating cartridge syringe which is inexpensive to manufacture, and efficient in operation.

Another object of the present invention is to provide a carpule that can deliver freshly mixed anesthetic and buffer solution, without remote pre-mixing or repeated mixing steps for subsequent administrations of anesthetic and buffer.

A further object of the present invention is to provide a carpule which provides adequately mixed solutions that are accurately measured and accurately proportioned in predetermined amounts and ratios, for administration from a single carpule.

Yet another object of the present invention is to provide a carpule which can reduce waste of buffered drug solution and increase efficiency by reducing the time and effort currently required to be expended by a health care professional to remotely pre-mix anesthetic and buffer solutions prior to administration of the mixture to a patient.

Still another object of the invention is to provide a carpule which can provide freshly mixed solutions and preventing decrease of pH of the solution over time when multiple, staggered injections are required during a lengthy medical procedure.

It is another object of the invention to provide a carpule having substantially standard size and shape that can be readily and easily inserted into the cartridge syringe barrel, whereby the anesthetic solution and buffer solution can be mixed together immediately before administration by actuation of a single plunger, and the mixture of anesthetic and buffer solutions administered to a patient without pre-mixing or remote mixing of the solutions.

Another object of the present invention is to provide a method of mixing buffered local anesthetic solution which can obviate the need for on-site, remote (away from the patient) mixing devices and methods for pre-mixing a buffered anesthetic solution prior to loading into a syringe for administration.

One object of the present invention is to provide a disposable carpule which may be readily and easily inserted into a cartridge syringe barrel configured for receiving and use of a conventional carpule, and which can be readily and easily removed therefrom after use, thereby requiring no additional pre-mixing steps as compared to the use of a conventional syringe.

Another object of the present invention is to provide a syringe device that can administer local anesthetics at, or near, physiological pH by controlling the pH of the solution exiting the mixing chamber from the syringe device.

These objects or advantages of the subject device and method, as well as other embodiments, objects or advantages not expressly provided, would be apparent and readily understood by the description and drawings provided herein.

### BRIEF DESCRIPTION OF THE DRAWINGS

FIG. 1 shows an embodiment of a carpule of the subject invention having a collapsible chamber disposed along an inner face of the carpule side wall, illustrating engagement of the sliding stopper with the tubular collapsible chamber. FIG. 1A is a perspective view of the carpule of the invention; FIG. 1B is a cross-sectional view of a carpule in accordance with the subject invention illustrating a valved top wall separating the mixing chamber and carpule reservoir; and FIG. 1C is a close-up or inset view of the sliding plunger engaged with the collapsible chamber in a manner which forces out contents of the collapsible chamber via a squeezing action.

FIG. 2 shows an embodiment of a carpule of the subject invention having a collapsible chamber disposed within the carpule, illustrating an embodiment having a folded collapsible chamber forming a pleated or accordion-like bellows. FIG. 2A is a perspective view of an embodiment of the carpule of the invention showing the sliding cap at a distal end of the carpule, prior to compression using a syringe plunger; FIG. 2B is a cross-sectional view of a carpule of the invention illustrating the mixing chamber partitioned by a valved partition, engaged with and in fluid communication with the folded embodiment of the collapsible chamber; FIG. 2C is a perspective view of an embodiment of the carpule of the invention showing the sliding cap moved proximally, compressing the collapsible chamber (not shown) and in contact with the distal end of the partition bounding the mixing chamber; FIG. 2D is a cross-sectional side view of and inset view of FIG. 2C, illustrating the compressed collapsible chamber.

FIGS. 3A-3C show views of an embodiment of a carpule of the subject invention having a partition forming a mixing chamber which can be punctured using at least one penetrating needle affixed to a pushrod extending through the fixed or capped stopper of the carpule. FIG. 3A is a side, cross-sectional view of a carpule of the invention comprising two penetrating needles for puncturing through the partition and into the reservoir and collapsible chamber; FIG. 3B is a close-up view of the mixing chamber at a proximal end of the carpule having penetrating needles for puncturing through the partition; FIG. 3C is the view shown in FIG. 3B, but showing the positional relation of a transfer needle used in conjunction with the subject carpule.

FIGS. 4A-4C show views of an embodiment of a carpule of the subject invention having a partition forming a mixing chamber, the partition comprising ports or apertures which can be aligned by a connecting pushrod extending through the fixed or capped stopper of the carpule causing fluid communication between the collapsible chamber and the mixing chamber, and fluid communication between the reservoir and the missing chamber. FIG. 4A is a side, cross-sectional view of a carpule of the invention comprising ports or apertures in the partition which can be rotated and aligned to open the collapsible chamber and reservoir into the mixing chamber; FIG. 4B is a close-up view of the mixing chamber at a proximal end of the carpule having an apertured partition, wherein the aperture is positioned to block fluid communication between the collapsible chamber and the mixing chamber; FIG. 4C is a close-up view of the mixing chamber at a proximal end of the carpule having an apertured partition, wherein the aperture is positioned to allow fluid communication between the collapsible chamber and the mixing chamber, and to allow fluid communication between the reservoir and the mixing chamber.

FIG. 5 shows and embodiment of a carpule of the subject invention comprising a collapsible chamber disposed within a reservoir of the carpule, wherein the contents of the collapsible chamber are expelled into the reservoir to mix with the contents thereof.

FIG. 6 shows an embodiment of the invention having a pleated or accordioned collapsible chamber disposed within the reservoir of a carpule, illustrating a distal end comprising a recessed slidable stopper and a proximal end comprising a tip configured to house a valve or rupturable membrane for expelling contents of the collapsible chamber into a mixing area positioned between the tip and the cap of the carpule.

FIGS. 7A-7D illustrate details of the embodiment shown in FIG. 6, specifically:

FIG. 7A shows a partial cross-sectional side view of the proximal end of a carpule having a collapsible chamber disposed within its reservoir, and illustrating a hollow conical, or inverted cup-shaped, tip which does not collapse during compression of the collapsible chamber, and which houses an optional one-way valve through which the contents of the collapsible chamber are expelled into a mixing area.

FIG. 7B shows a partial perspective view of the proximal end of a carpule having a collapsible chamber disposed within its reservoir, and illustrating a hollow conical or inverted cup-shaped tip which houses a one-way valve through which the contents of the collapsible chamber are expelled into a mixing area.

FIG. 7C shows a side cross-sectional view of a carpule having a collapsible chamber disposed within its reservoir, and illustrating the features: a conical or inverted cup-shaped proximal tip with a one-way valve and mixing area; support rings intermittently positioned on the outer surface and along the length of the collapsible chamber, and a recessed distal plunger.

FIG. 7D shows perspective view of a carpule having a collapsible chamber disposed within its reservoir, and illustrating the support rings formed on an outer surface of the collapsible chamber, and a recessed distal sliding stopper or plunger.

The drawings presented are not to scale.

### DETAILED DESCRIPTION OF THE INVENTION

The subject invention concerns a carpule and a system and method particularly useful for mixing and delivering buffered anesthetic to a patient without requiring remote mixing of the anesthetic and buffer prior to administration. The invention includes use of the carpule in a conventional cartridge syringe, preferably a conventional aspiration cartridge syringe.

The subject invention comprises a carpule having a reservoir for containing a drug solution, e.g., a local anesthetic solution, such as lidocaine or its pharmaceutical salt, with or without epinephrine, wherein the reservoir has disposed therein a collapsible chamber for containing a second solution, e.g., a buffer solution, such as sodium bicarbonate solution.

The carpule of the subject invention comprises at least:
a primary reservoir for containing and storing drug solution,
a discrete collapsible chamber within the reservoir, for containing and storing a second solution, such as buffer solution, separate and apart from the solution contained within the primary reservoir, and
a mixing area, which can optionally be a separate mixing chamber, for receiving solutions from the distal reservoir and collapsible chamber.

A mixing area or mixing chamber can be formed by a partition positioned substantially horizontally, generally perpendicular to the longitudinal axis of the carpule, and providing a space or cavity capable of receiving for receiving solutions from both the reservoir and the collapsible chamber, allowing for mixing of the solutions before their administration to a patient. The partition can comprise a rupturable membrane which is pressure sensitive and ruptures when pressure is applied using the plunger or piston of the syringe, or can comprise a one-way valve which opens when pressure is applied using the plunger or piston of the syringe, allowing the contents of the reservoir and collapsible chamber to empty into the mixing area or mixing chamber. In an embodiment comprising a mixing area, the partition can comprise a rupturable membrane or can comprise at least one valve. In an embodiment comprising a separate mixing chamber, the partition can include at least two one-way valves.

The collapsible chamber is generally cylindrical or tubular in shape and is hollow, whereby the collapsible chamber can be compressed and flattened along its length, or folded onto itself in an accordion-like fashion where the distal end of the collapsible chamber is compressed toward the proximal end of the collapsible chamber. Typically, compression of the collapsible chamber is effected by actuation of the syringe plunger which engages and displaces a sliding stopper or sliding plug of the carpule. As the sliding plug is displaced, it contacts the collapsible chamber in a manner which unidirectionally expels contents from the collapsible chamber and, simultaneously, the carpule reservoir.

Operation of the system of the invention can employ a conventional cartridge syringe or can employ a syringe modified to receive a carpule configured to comprise a collapsible chamber and, optionally, a mixing chamber as described herein.

All references to a “syringe” used herein, refer to a cartridge syringe or an aspiration cartridge syringe for use with a single carpule.

For purposes of the subject invention, the terms “distal” or “superior” and “proximal” and “inferior” are used to describe the relative position of the components of the carpule or system. “Distal” or “superior” refers to the end farthest away from the patient when the syringe is used, i.e., the end from which the user of the syringe actuates the plunger; “proximal” or “inferior” refers to the end closest to the patient when in use, i.e., the end of the syringe engaged with a hypodermic needle.

The terms “axial,” “longitudinal,” or “longitudinal axis,” or “axial length” all refer to a relative position or direction along one or more long axis or length dimension of the generally cylindrical syringe and carpule of the invention.

The subject invention further comprises a method of administering a mixture of anesthetic solution and buffering solution, forming a mixed, buffered anesthetic solution by employing a novel carpule as described and shown herein.

Advantages of the present invention include convenience of use and greater efficiency in a clinical setting for injectable drugs, such as anesthetics, which are commonly remotely mixed with another solution immediately prior to administration. This advantage is achieved by providing a carpule in accordance with the subject invention comprising a reservoir for containing anesthetic solution and a buffering solution in a collapsible chamber disposed within the reservoir.

The carpule of the subject invention therefore can provide a self-contained anesthetic/buffer solution mixing and delivery system which does not require pre-mixing of the anesthetic and buffer prior to loading the syringe with anesthetic or buffer solution, nor does it require mixing to be conducted remotely or outside the syringe or system of the invention.

In addition, the carpule and system of the subject invention can provide unexpected advantages, reducing patient discomfort, carpule damage, and inconsistent dosing by delivering buffered drug solution using an optimal force. If the force required varies significantly during administration, patient discomfort can increase, carpule damage risk is increased, and accurate drug delivery and consistent dosing can become a challenge.

Plunger force is the amount of pressure required to push the syringe plunger down in a prefilled carpule, using a manual piston syringe, effectively squeezing out the drug solution. This seemingly simple action, however, can have a profound impact on the entire drug delivery process.

Factors affecting the amount of pressure required to be applied to the sliding stopper/plunger provided in the carpule, and to deliver drug from a carpule using a cartridge syringe, include the viscosity of the solutions held within the reservoir and chamber, as well as the break-loose force and gliding force required by the plunger or sliding stopper component. A conventional sliding stopper/plunger used in a carpule is made of a halobutyl rubber material. Because this material can be susceptible to deterioration by acidic or alkaline solutions, it is preferred to include a coating on it outer surface. A preferred coating can comprise a fluoropolymer or silicone elastomer. These coatings can be sprayed onto the outer surface of the plunger or sliding stopper. The coating can prevent deterioration, and can advantageously optimize the force required to initiate movement (the “break-loose force”) and to move the sliding stopper (the “gliding force”) for administering the drug solution.

The coating can also be applied to the collapsible chamber to maintain stability of the drug solution within the reservoir or the buffer solution held within the collapsible chamber.

Acceptable ranges of force required to administer the drug solution can vary, but can be:
8-10N (Newton): A preferred range of force required to actuate the plunger in the prefilled carpule. This is the preferred force needed for smooth and controlled drug delivery.
11-15N: An acceptable range of force, meaning that a force up to 15N can be applied without compromising functionality or safety.
16-21N: This range represents the absolute maximum force to be applied before there risking damage to the carpule or negatively affecting drug delivery.

The subject invention can provide oral healthcare practitioners with a preferable break-loose force of about 10N, with 14-15N being an acceptable value, and 20-21N being the absolute maximum. For gliding forces, the values are mostly dependent on drug viscosity, needle size, collapsible chamber, and the impact of the drug on the barrel.

Regarding viscosity of the drug solution within the reservoir, a carpule of the subject invention can facilitate administration using 2% Lidocaine HCl (viscosity of about 1.5 cP), 2% Lidocaine HCl with epinephrine (viscosity about 1.6 cP), or 4% Septocaine™ (Articaine HCl) (viscosity of about 2.0 cP).

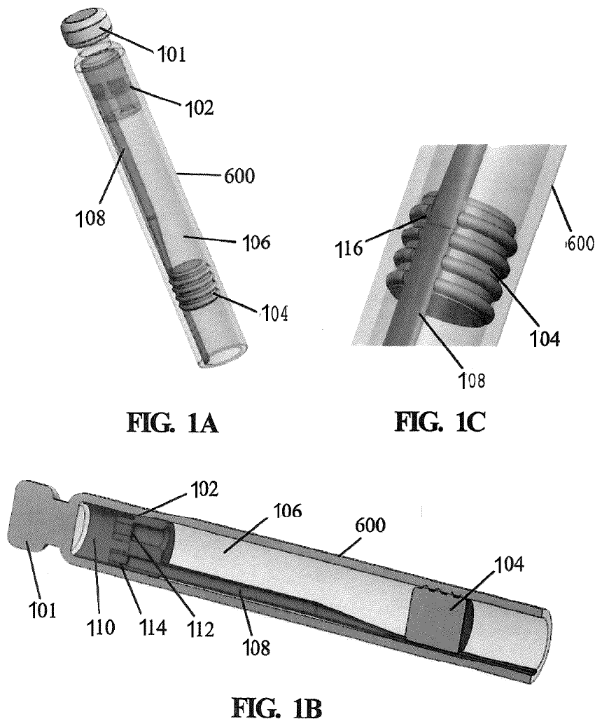

Turning to the drawings provided herewith, certain embodiments of a carpule in accordance with the subject invention are exemplified and shown. FIGS. 1A, 1B, and 1C show an embodiment of a carpule of the subject invention having a tubular collapsible chamber which is disposed along an inner face of the carpule side wall, and flattens upon engagement with the sliding plug or stopper.

As illustrated in FIGS. 1A-1C, a carpule of the invention comprises a primary reservoir for containing and storing drug solution, a discrete collapsible chamber within the reservoir for separately containing and storing a second solution, and a mixing chamber for receiving solutions from the reservoir and collapsible chamber, allowing for mixture of the solutions and expulsion of the mixture therefrom.

FIG. 1A is a perspective view of the carpule of the invention. Specifically, FIG. 1A is a perspective view of the carpule 600 of the invention comprising end stoppers or plugs, namely proximal fixed stopper 101 and a distal sliding plug 104. Within the carpule 600 are the valved mixing chamber 102. Bounded by the mixing chamber 102 and sliding plug 104 is reservoir 106 for containing and storing a drug solution. Also illustrated is an embodiment of the collapsible chamber 108 disposed longitudinally along an inner face of the carpule side wall, extending substantially the entire length of the carpule from the proximal side of the sliding stopper or plunger at the distal end of the carpule to the shouldered portion of the carpule at its proximal end.

FIG. 1B is a cross-sectional view of the carpule shown in FIG. 1A, in accordance with one embodiment of the subject invention. FIG. 1B illustrates unidirectional valves 112 and 114 in valved partition 102 allowing the reservoir 106 and collapsible chamber 108 to be independently in fluid communication with the mixing chamber 110. Valve 112 allows unidirectional fluid communication between the reservoir 106 and mixing chamber 110, and valve 114 allows unidirectional fluid communication between the collapsible chamber 608 and mixing chamber 110.

FIG. 1C is a close-up or inset view of the sliding plunger engaged with the collapsible chamber in a manner which forces out contents of the collapsible chamber via a compressing or squeezing action. FIG. 1C shows sliding plug 104 within the carpule 600 of FIG. 1A. Also illustrated is groove 116 configured close and compressing engagement with collapsible chamber 108, allowing for passage of the sliding plug move over the collapsing chamber.

As shown, the mixing chamber is formed by a valved partition positioned horizontally, perpendicular to the longitudinal axis of the carpule, and provides a cavity having a size optimized for mixing of the solutions before administration of the mixture to a patient.

The sliding stopper can comprise a groove configured to hold the collapsible chamber in an optimal position, i.e., reduce movement of the collapsible chamber such that it is inefficiently collapsed and emptied. The groove can also serve to facilitate compression or squeezing action applied to the collapsible chamber, allowing passage of the sliding plug over the collapsible chamber only when the collapsible chamber is in a compressed or collapsed configuration.

In use, when the syringe plunger is depressed, the sliding stopper contacts the inside wall of the carpule, compressing the collapsible chamber, expelling the contents therefrom unidirectionally through the valved partition and into the mixing chamber. At the same time, solution contained within the reservoir of the carpule is unidirectionally forced through a separate valve in the valved partition and into the mixing chamber.

As the plunger is more fully depressed, the content of the mixing chamber, which is now a mixture of the content within both the first and second reservoirs, e.g., anesthetic and buffer, is expelled from the syringe and administered to a patient.

FIGS. 1A-1C also illustrate the process of administering the contents from the partitioned carpule 600 of the invention, wherein FIGS. 1A and 1B show the collapsible, second reservoir 108 containing fluid on one side of the plunger 104 and being collapsed on the other side. FIG. 1C shows the plunger 104 collapsing the second reservoir and the groove 116 allowing passage of the collapsed second reservoir 108 through plunger 104.

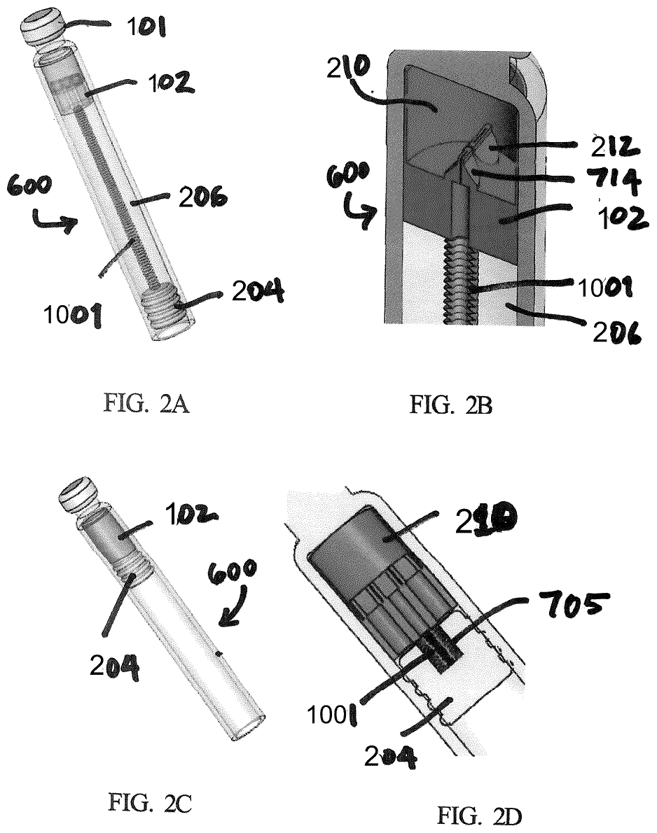

FIGS. 2A, 2B, 2C, and 2D show an embodiment of a carpule of the subject invention having a collapsible chamber disposed within the carpule. FIGS. 2A, 2B, 2C, and 2D illustrate an embodiment comprising a collapsible chamber which is folded or pleated in the form of an accordion-like bellows.

FIG. 2A is a perspective view of an embodiment of a carpule 600 of the invention showing the sliding plug 204 at a distal end of the carpule, prior to compression using a syringe plunger. Engaged with sliding plug 204 is collapsible chamber 1001, shown in expanded configuration and centered within reservoir 206 of carpule 600. For ease of reference, also illustrated is valved partition 102 and fixed proximal stopper 101, as also shown in FIGS. 1A-1C.

FIG. 2B is a cross-sectional and close-up view of a valved partition 102 within a reservoir of carpule 600 of the invention. Illustrated in FIG. 2B are mixing chamber 210 formed by a valved partition 102 which engages with and is in fluid communication with the folded embodiment of the collapsible chamber 1001. Contents of reservoir 206 are in fluid communication with mixing chamber 210 via valve 212; contents of collapsible chamber 1001 (shown in in its expanded configuration) are in fluid communication with mixing chamber 210 via valve 714.

FIG. 2C is a perspective view of an embodiment of the carpule 600 as shown in FIG. 2A, but showing sliding plug 204 moved proximally, compressing the folded collapsible chamber (not shown) and in contact with the distal end of the valved partition 102 bounding the mixing chamber.

FIG. 2D is a cross-sectional and close-up cross-sectional view of the embodiment depicted in FIG. 2C, illustrating collapsible chamber 1001 in compressed configuration. FIG. 2D also illustrates an embodiment wherein sliding plug 204 comprises a recess 705 formed in its proximal face, configured to allow space for and matingly engage compressed collapsible chamber 1001.

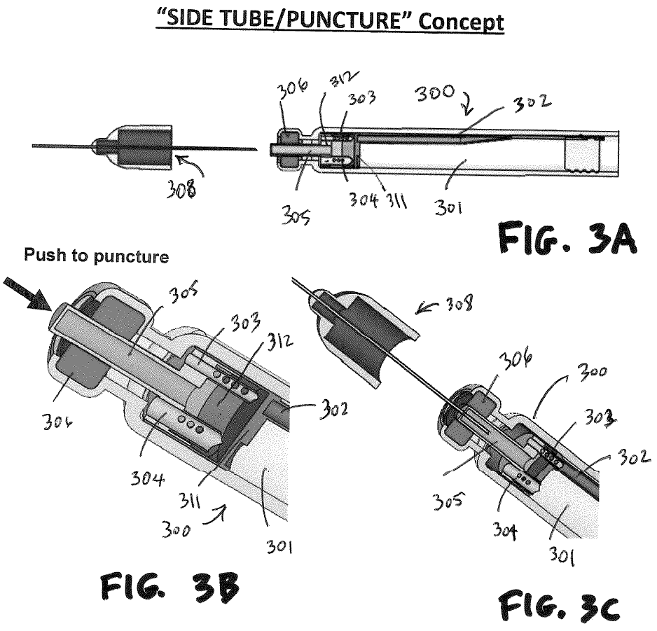

FIGS. 3A-3C show views of an embodiment of a carpule of the subject invention having a partition forming a mixing chamber which can be punctured using at least one penetrating needle affixed to a pushrod 305 extending through the fixed or capped stopper 306 of the carpule. FIG. 3A is a side, cross-sectional view of a carpule 300 of the invention comprising two penetrating needles 303 and 304 for puncturing through the partition 311 and into the reservoir 301 and collapsible chamber 302; FIG. 3B is a close-up view of the mixing chamber 312 at a proximal end of the carpule having penetrating needles 303 and 304 for puncturing through the partition 311; FIG. 3C is the view shown in FIG. 3B, illustrating the positional relation of a transfer needle 308 used in conjunction with the subject carpule 300.

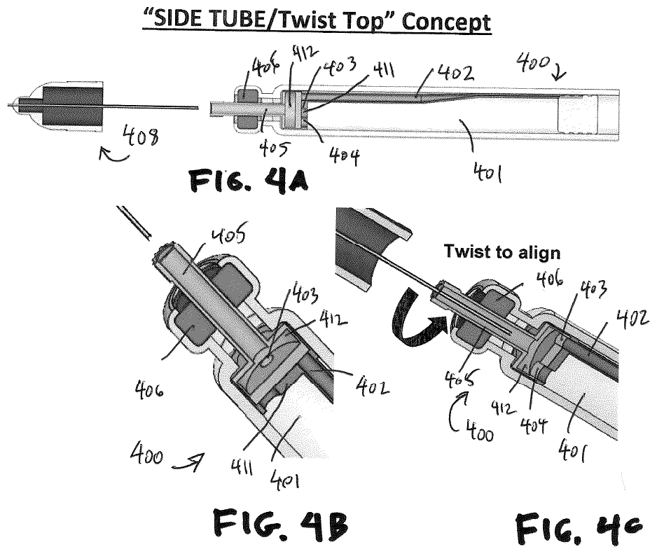

FIGS. 4A-4C show views of an embodiment of a carpule 400 of the subject invention having a partition 411 forming a mixing chamber 412, the partition comprising ports or apertures 403, 404 which can be aligned by a connecting pushrod 405 extending through the fixed or capped stopper 406 of the carpule causing fluid communication between the collapsible chamber 402 and the mixing chamber 412, and fluid communication between the reservoir 401 and the mixing chamber 412. FIG. 4A is a side, cross-sectional view of a carpule 400 of the invention comprising ports or apertures 403, 404 in the partition 411 which can be rotated and aligned to open the collapsible chamber 402 and reservoir 401 into the mixing chamber; FIG. 4B is a close-up view of the mixing chamber 412 at a proximal end of the carpule 400 having an apertured partition 411, wherein the aperture 403 is positioned to block fluid communication between the collapsible chamber 402 and the mixing chamber 412; FIG. 4C is a close-up view of the mixing chamber 412 at a proximal end of the carpule 400 having an apertured partition 411, wherein the aperture 403 is positioned to allow fluid communication between the collapsible chamber 402 and the mixing chamber 412, and wherein the aperture 404 is positioned to allow fluid communication between the reservoir 401 and the mixing chamber 412.

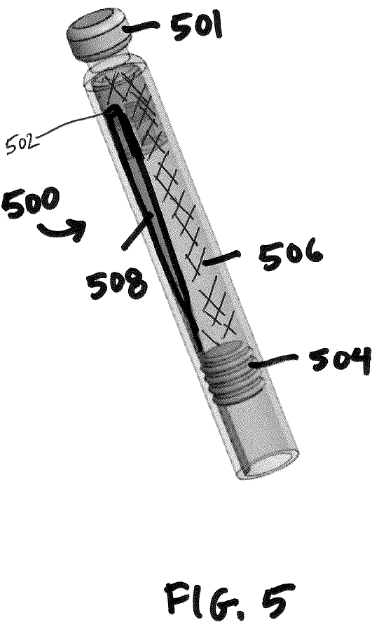

FIG. 5 shows and embodiment of a carpule 500 of the subject invention comprising a collapsible chamber 508 disposed within a reservoir 506 of the carpule 500, wherein the contents of the collapsible chamber 508 are expelled into the reservoir 506 to mix with the contents thereof. This embodiment of the carpule does not comprise a mixing chamber separate and apart from the reservoir or collapsible chambers. The reservoir 506 contains a collapsible chamber 508 having a sealed proximal end 502 wherein, under pressure, the sealed end breaks, allowing contents of the collapsible chamber 508 to be expelled directly into the reservoir 506 to mix with the reservoir contents before being administered through capped end 501 to a patient.

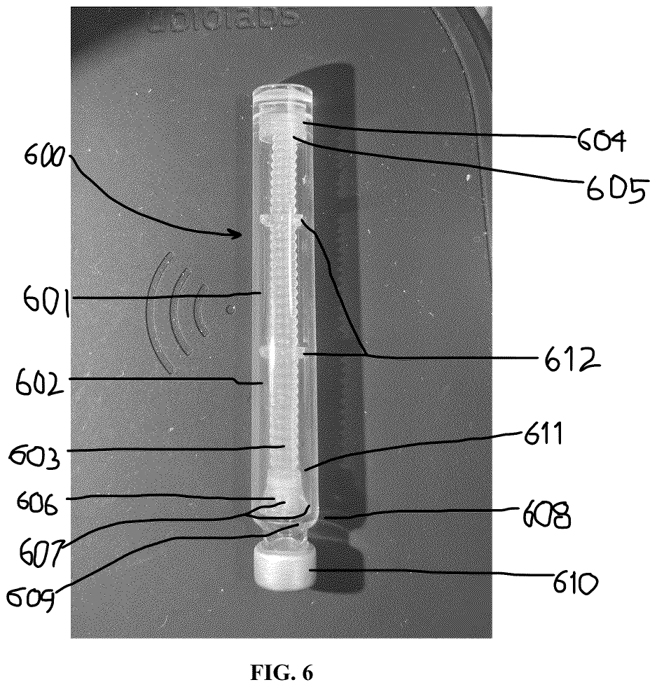

FIG. 6 shows a preferred embodiment of a carpule 600 of the invention comprising a tubular side wall 601 enclosure forming a reservoir 602 within the bounds of the side wall. Drug solution can be filled within the reservoir 602, but is not filled within collapsible chamber 603, which contains buffer solution. Collapsible chamber 603 has a pleated or accordioned configuration which can collapse down upon itself as pressure is applied to a cartridge syringe piston (not shown) and thereby, a sliding stopper or plunger 604, provided at a distal end of the carpule and collapsible chamber.

A preferred configuration of the sliding stopper or plunger has a recess 605 in its proximal face for receiving the collapsible chamber. This recess can advantageously stabilizes the collapsible chamber and can maximize the volume of drug solution that can be filled within the reservoir 602.

At a proximal end of the collapsible chamber, a hollow conical or inverted cup-shaped tip 606 can be provided. The outer edge of the conical tip engages the inner face of the shoulder 608 formed at the proximal end of the carpule. The conical tip can also be useful to stabilize the collapsible chamber. A seal can be provided at the interface of the conical tip and the collapsible chamber to prevent emptying of the contents of the collapsible chamber prior to use. The seal can be a pressure-sensitive, rupturable membrane which can be broken by pressure forces when the plunger is actuated. Alternatively, the seal can comprise a one-way valve which opens and releases the contents of the collapsible chamber when pressure is applied to the plunger.

In one preferred embodiment, the hollow conical tip can include one or more ports or vents 607, which allows solution released from the collapsible chamber to flow into the reservoir, thereby facilitating mixing of the drug solution and buffer solution during use. The conical tip is preferably stationary and is held into position at the carpule shoulder such that a space is formed, providing a mixing area 609, proximal to the collapsible chamber, and distal to the end cap 610. The mixing area therefore includes the area within the hollow conical tip.

The conical tip can be formed having a recess 611 formed at its distal end to receive and engage the collapsible chamber. Optionally, one or more ribs 612 can be provided along the length of the collapsible chamber. These one or more ribs can stabilize the collapsible chamber when pressure is applied during use by preventing bending of the tube, and can be coated to reduce friction between the collapsible chamber and the inner side wall of the carpule, thereby facilitating the movement and reducing the gliding force required to actuate the plunger.

The collapsible chamber, recessed plunger or sliding stopper, conical tip and optional ribs, can be molded as a single unit for inserting into the carpule, or each component can be separately formed and assembled for use.

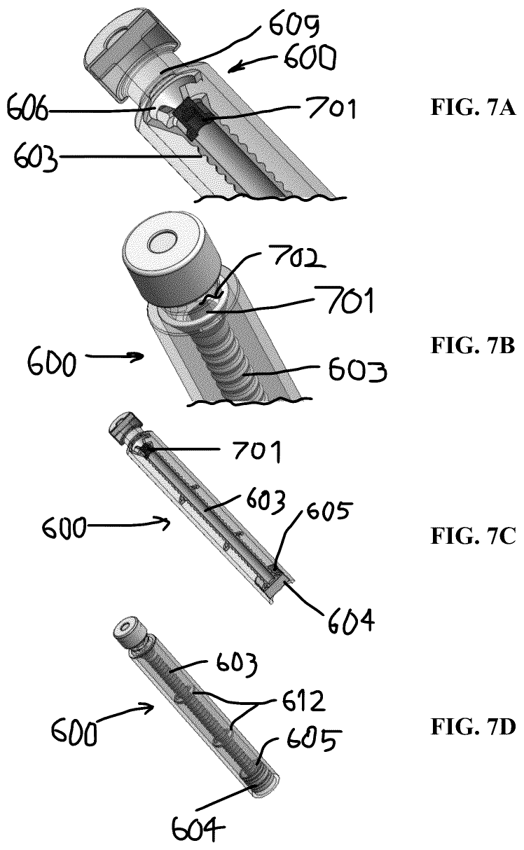

FIGS. 7A-7D illustrate details of the embodiment shown in FIG. 6. FIG. 7A shows a partial cross-sectional side view of the proximal end of a carpule 600 having a collapsible chamber 603 disposed within its reservoir, and illustrating a vented conical shaped tip 606. In this embodiment, the collapsible chamber is sealed using a one-way valve (as described and shown herein) through which the contents of the collapsible chamber are expelled into mixing area 609.

FIG. 7B shows a partial perspective view of the proximal end of a carpule 600 having a collapsible chamber 603 disposed within its reservoir, as shown in FIG. 7A, and illustrating the positioning of the hollow conical shaped tip engaged with the inner wall at the shoulder formed in carpule 600, and the positioning of the one-way valve 701 provided at the interface of the collapsible chamber and the hollow conical tip, through which the contents of the collapsible chamber are expelled into a mixing area 702.

FIG. 7C shows a side cross-sectional view of a carpule 600 having a collapsible chamber 603 disposed within its reservoir, and illustrating the features of: a conical shaped proximal tip with a one-way valve 701, and a recess 605 provided in sliding stopper or plunger 604.

FIG. 7D shows perspective view of a carpule 600 having a collapsible chamber 603 disposed within its reservoir, and illustrating the support ribs 612 formed on an outer surface of the collapsible chamber, and a recess 605 provided in sliding stopper or plunger 604.

It would be understood by ordinarily skilled artisans that the design or configuration of certain components described herein can be modified or optimized. For example, larger diameter carpule barrels offer more space for the drug to spread, reducing resistance and the required force. Conversely, slimmer barrels concentrate the fluid, potentially increasing the needed pressure. A flat geometric shape of the proximal face of the sliding plunger can create more friction against the barrel wall, requiring more force. A rounded or beveled tip, however, can smooth the glide, reduce pressure requirements. Similarly, the shape or configuration of the pores or vents provided in the hollow conical tip can facilitate the mixing, and affect the amount of pressure required to actuate the plunger.

Friction can be reduced by use of low-friction materials, such as silicone or fluoropolymer coatings on the plunger and collapsible chamber. Reduced frictional resistance can provide smoother movement and lower force requirements.

In addition, by using a spray-on fluoropolymer coating on the collapsible chamber and sliding plunger, the surface can be made compatible with different anesthetic drugs. Silicone coating can eliminate direct contact between the halobutyl rubber material forming the sliding plunger, and the barrel material. This can prevent potential drug-rubber interactions, leaching, and particle formation, crucial for drug stability and purity.

The sealing ribs provided along the length of the collapsible chamber can create an interference fit with the barrel's inner diameter to ensure drug containment and prevent leakage during storage and use.

It is understood that the mixing chamber can optionally include, but does not require, a turbulence-enhancing apparatus disposed therein to promote and provide homogenous mixing of the drug and buffer solutions. Alternatively, the mixing chamber can be present without a turbulence-enhancing apparatus disposed therein.

When present, the turbulence-enhancing apparatus disposed within the mixing chamber serves to create turbulent flow of the solutions entering the mixing chamber from the carpule reservoir and collapsible chamber, resulting in thorough mixing of the solutions immediately prior to administration of the then-buffered anesthetic solution through the hypodermic transfer needle.

In one preferred embodiment, the turbulence-enhancing apparatus is an inert material separate and distinct from the mixing chamber, and is not formed as an integral part of the mixing chamber, i.e., it is a distinct apparatus that is not etched or formed into the wall of the mixing chamber. A preferred turbulence-enhancing apparatus can be made of plastic or metal which does not interact chemically or physically with the buffered anesthetic solution and can be provided in a twisted or convoluted or highly porous configuration, e.g., steel wool, to facilitate turbulent flow and mixing of the solutions.

Alternatively, the turbulence-enhancing apparatus can be a finned disc or turbine which can facilitate mixing of the solutions by a rotational motion activated by fluid passing by the apparatus. The mixing chamber comprising the turbulence-enhancing apparatus preferably provides a substantially homogeneous mixture of buffer and anesthetic solutions.

The mixing chamber preferably comprises a turbulence-enhancing apparatus disposed therewithin and separate from the mixing chamber itself, which serves to create turbulent flow of the solutions entering the mixing chamber from the partitioned carpule reservoirs, resulting in thorough mixing of the solutions immediately prior to administration of the buffered anesthetic solution through the delivery port and hypodermic transfer needle. In one preferred embodiment, the turbulence-enhancing apparatus is an inert material separate and distinct from the mixing chamber, and is not formed as an integral part of the mixing chamber, i.e., it is a distinct apparatus that is not etched or formed into the wall of the mixing chamber.

A preferred turbulence-enhancing apparatus can be made of plastic or metal which does not interact chemically or physically with the buffered anesthetic solution and can be provided in a highly twisted or convoluted or highly porous configuration, e.g., steel wool, to facilitate turbulent flow and mixing of the solutions.

The mixing chamber comprising the turbulence-enhancing apparatus preferably provides a substantially homogeneous mixture of buffer and anesthetic solutions.

The present invention provides advantages over prior devices in, at least the following ways:
it allows for mixing and dispensing local anesthetic and buffering solution from a single syringe using prefabricated carpules dispensed in a single actuating step; it provides a method of mixing an anesthetic solution with a buffering solution which provides a convenient and all-in-one step of producing a thorough and precise mixture of buffered anesthetic solution;
it does not require any activation step; therefore, it is better suited for practicality and efficiency in a clinical setting.
it provides a method of mixing buffered local anesthetic solution which will obviate the necessity of any mixing steps prior to loading into a syringe for administration;
it provides a carpule having substantially standard size and shape which may be readily and easily inserted into a conventional syringe barrel for use, and readily and easily removed therefrom after use;
it provides a disposable readily and easily inserted carpule comprising a reservoir prefilled with local anesthetic solution and a collapsible chamber prefilled with sodium bicarbonate solution which are readily available for mixing or dispensing;
it provides a carpule and system which facilitates dispensing of buffered local anesthetic solution instantly prior to administering the solution into the intended target tissue;
it provides a single carpule and system for simultaneously transferring solutions in preset ratios and amounts from a reservoir and collapsible chamber directly into a mixing chamber;
it can provide a mixing chamber causing turbulence between the two solutions contained within a single carpule; and
it provides a unique carpule configuration having the same length and diameter as a standard carpule but having a drug (e.g., local anesthetic) solution and buffer (e.g., sodium bicarbonate) solution within a single carpule at a pre-set and accurately measured and reproducible ratio of at least 8:1 up to a ratio of 10:1, and preferably a ratio of 9:1. The ratio of 9:1 results in a final pH closely approximating physiological pH of 7.4 when using standard 8.4 % sodium bicarbonate as buffer solution with standard 2% lidocaine HCl with 1,000,000 epinephrine.

Chemically, amide local anesthetics are weak bases. Commercial local anesthetic cartridges are purposefully formulated as relatively acidic solutions to enhance the solubility of the anesthetic salts and to prolong shelf life. Typically, commercial 2% lidocaine solutions with 100,000 epinephrine have a pH of about 3.93±0.43. The pH of the solution also affects the way the anesthetic works. Like most injectable local anesthetics, lidocaine with epinephrine solution contains two forms of the anesthetic salt:
the uncharged, de-ionized, or “active” free base form, which is lipid soluble; and
the charged or ionized cationic form, which is not lipid soluble.

The de-ionized form more readily penetrates the nerve membrane to enter the nerve axon, where the anesthetic attaches to receptors on the sodium channels, resulting in a blockade of nerve conduction. According to the Henderson-Hasselbalch equation, in any sample of local anesthetic solution the ratio of the de-ionized species of the anesthetic to the ionized species of the anesthetic is based on the pH of the sample. At a more acidic pH, the ionized cationic form predominates. For instance, at a pH of 3.9, a typical cartridge of lidocaine with epinephrine contains only 1 molecule of de-ionized anesthetic for every 10,000 molecules of ionized anesthetic. On the other hand, closer to physiologic pH, more de-ionized anesthetic is present. For instance, at the physiologic pH of 7.4 there is one molecule of de-ionized lidocaine in solution for every 4 molecules of ionized lidocaine. At the physiologic pH, then, there is 2,500× more of the active form of the anesthetic available than at pH 3.93±0.43.

In use, the subject invention employs a conventional carpule-holding syringe, except that the carpule is includes a reservoir and a collapsible chamber, wherein the novel-configured carpule can be loaded within a cartridge syringe barrel prior to administration of a medicament.

Specifically, the method comprises the steps of:
a) providing a syringe having a syringe barrel capable of receiving a single carpule, as described herein;
b) loading the carpule containing a drug solution and buffer solution into the syringe barrel; and
c) actuating, in a single step, the plunger of the cartridge syringe to actuate the sliding plug in the carpule, causing the reservoir to expel and deliver anesthetic solution and causing the collapsible chamber to compress and thereby expel and deliver buffering solution into a mixing chamber within the carpule, whereby the anesthetic solution and buffering solution are mixed, preferably homogeneously, and delivered from the mixing chamber and into the hypodermic transfer needle of the syringe and into a target tissue.

The method can further include removing the expended carpules from the syringe barrel and repeating the above steps a) through c) as needed.

It is contemplated that the invention provides a significant role in ease-of-use and efficiency in administering local anesthetic buffered solution in a pH control fashion. The present invention provides optimal dosing control of freshly buffered local anesthetic solution at the fingertips of medical practitioners.

It is to be understood that certain descriptions of the invention have been simplified to focus on elements that are relevant for a clear understanding of the invention, while eliminating, for purposes of clarity, other elements that those of ordinary skill in the art will appreciate may also comprise a portion of the invention. While the present invention has been presented in accordance with the several preferred and practical embodiments thereof, it is recognized that departures from and variants of the instant disclosure are contemplated as being fully within the spirit and scope of the invention.

The above disclosure and example generally describe the present invention and is provided for purposes of illustration and is not intended to limit the scope of the invention.

The invention described herein may be practiced in the absence of any element or elements, limitation or limitations which is not specifically disclosed herein. Thus, for example, in each instance herein, any of the terms “comprising,” “consisting essentially of,” and “consisting of” may be replaced with either of the other two terms. The terms and expressions are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the claims.

1. A carpule for use with a cartridge syringe comprising:
a primary reservoir for containing and storing drug solution, said reservoir bounded by a cylindrical or tubular carpule sidewall, a proximal end plug, and a distal sliding stopper or plunger,
a discrete collapsible chamber disposed within the reservoir, for containing and storing a second solution separate and apart from the solution contained within the primary reservoir,
wherein the collapsible chamber is disposed within the reservoir such that a mixing area is provided for receiving solutions expelled from the primary reservoir and collapsible chamber, and allowing mixing of the solutions before administration to a patient.

2. The carpule of claim 1, wherein the collapsible chamber is a tube or cylinder abutting an inner face of the carpule side wall, the collapsible chamber having a sealed distal end and sealed proximal end, wherein the proximal end seal comprises a rupturable membrane or one or more unidirectional valve opened by applied pressure when the cartridge syringe is actuated to move the sliding stopper or plunger, thereby flattening the collapsible chamber and expelling the second solution into the mixing area or mixing chamber at the proximal end of the carpule reservoir.

3. The carpule of claim 1, wherein the collapsible chamber is configured as a pleated or accordioned tubular bellows disposed along a central longitudinal axis of the carpule, said pleated or accordioned collapsible chamber having a sealed distal end and sealed proximal end, wherein the proximal end seal comprises a rupturable membrane or one-way valve opened by applied pressure when the cartridge syringe is actuated to move the sliding stopper or plunger, thereby collapsing the collapsible chamber by compressing the sealed distal end of the collapsible chamber toward the proximal end of the collapsing chamber and expelling the second solution into the mixing area or mixing chamber at the proximal end of the carpule reservoir.

4. The carpule of claim 1 wherein the mixing area or mixing chamber is a discrete mixing chamber having a valved partition, wherein the mixing chamber is in fluid communication with the primary reservoir through a first unidirectional valve provided in the valved partition, and wherein the mixing chamber is in fluid communication with the collapsible chamber through a second unidirectional valve provided in the valved partition.

5. The carpule of claim 4 wherein the mixing chamber comprises a turbulence-enhancing apparatus disposed therewithin and wherein the turbulence enhancing apparatus is not integral with the mixing chamber.

6. The carpule of claim 1 wherein the collapsible chamber disposed within the reservoir is coated with an inert coating agent selected from silicone and fluoropolymer.

7. The carpule of claim 3, wherein the sliding stopper or plunger has a distal and face and a distal face, wherein the distal face is substantially flat or domed and the proximal face includes a recess for matingly receiving the distal end of the collapsible chamber,

8. The carpule of claim 3, further comprising a conical or inverted cup-shaped member having a distal face and a proximal face, wherein said distal face of the conical member includes a recess to matingly receive the proximal end of the collapsible chamber, and wherein said proximal face of the conical member forms a ring which engages a shouldered internal sidewall of the carpule, thereby providing a mixing area proximal to the conical member and distal to the proximal end plug.

9. The carpule of claim 8, wherein the conical member comprises at least one port or vent allowing solution expelled from the collapsible chamber to mix with solution in the reservoir.

10. A method for mixing anesthetic solution and buffer solution and concomitantly administering mixed anesthetic solution and buffer solution to a patient in need thereof, said method comprising:
providing a carpule of claim 1
prefilling the collapsible chamber with buffer solution and prefilling the reservoir with anesthetic solution outside the collapsible chamber,
inserting the carpule into a cartridge syringe for use, and
actuating the cartridge syringe to concomitantly compress the collapsible chamber, causing mixing of the buffer solution and anesthetic solution in the mixing area and
administering the mixed buffer solution and anesthetic solution to the patient.

11. The method of claim 10, wherein the collapsible chamber is a pleated or accordioned tube or cylinder.

12. The method of claim 10, wherein the buffer solution is sodium bicarbonate.

13. The method of claim 10, wherein the anesthetic solution comprises lidocaine.

14. The method of claim 13, wherein the lidocaine is lidocaine with epinephrine.

15. The method of claim 10, wherein the buffer solution and the anesthetic solution has a pH of about 7.4 when mixed.

16. A system for pre-mixing anesthetic and buffer solutions and administering the pre-mixed solutions to a patient, said system comprising a cartridge syringe and a carpule of claim 1.

17. The system of claim 16, wherein the collapsible chamber is a pleated or accordioned tube or cylinder.

18. The system of claim 16, wherein the carpule is pre-filled with buffer solution in the collapsible chamber, and is pre-filled with anesthetic solution in the reservoir and outside the collapsible chamber.

19. The method of claim 16, wherein the buffer solution is sodium bicarbonate.

20. The method of claim 16, wherein the anesthetic solution is lidocaine with epinephrine.
